(19) United States
(12) Patent Application Publication
(10) Pub. No.: US 2009/0157177 A1
(43) Pub. Date: Jun. 18, 2009

(54) Sewing Ring for a Prosthetic Tissue Valve

(76) Inventor: Robert G. Matheny, Norcross, GA (US)

Correspondence Address: Ballard Spahr Andrews & Ingersoll, LLP, SUITE 1000, 999 PEACHTREE STREET, ATLANTA, GA, 30309-3915, US

(21) Appl. No.: 11/958,407

(22) Filed: Dec. 18, 2007

(51) Int. Cl. A61F 2/24

(52) U.S. Cl. 623/2.41

(57) ABSTRACT

The invention provides a trileaflet semi-lunar prosthetic tissue valve for aortic, pulmonary, mitral, or tricuspid valve replacement. The valve is planar before attachment at an annulus of a valvular lumen, and non-planar upon attachment at the annulus of the defective valve. A sewing ring having a circumference greater than the annular circumference of annulus of the valve being replaced is also described and the sewing ring is placed at the approximate position of the annulus of the defective valve in a non-planar configuration.

### FIELD OF THE INVENTION

The invention is a sewing ring for a prosthetic tissue valve. The sewing ring is designed to fit securely within the annulus of the replaced valve and to overcome problems that exist with other artificial valves at the point where the sewing articulates the device in the valve annulus.

### BACKGROUND OF THE INVENTION

Two basic types of artificial heart valves are used to replace defective heart valves: mechanical valves and tissue valves. In addition, research and experimentation is being done to develop valves that can be placed in the patient percutaneously without open heart surgery.

Mechanical valves, while quite durable, have the deficiency of requiring open heart surgery, risk peri-valvular leakage on the outside of the valve between the valve and the attachment lumen, and require a lifetime of administration of anti-coagulants which administration requires close (usually bi weekly) monitoring in order to avoid either bleeding or clotting stroke. Mechanical valves also risk development of stenosis at the valve replacement site, and incur chronic hemolysis (damage to red blood cells by the mechanical action of the valve).

Tissue valves typically last from 10 to 15 years in less active (elderly) adults and are of porcine or human origin. They fail because the tissue begins to wear, commensurate with the fact that the valves are retrieved after already having undergone partial lifetimes of use. Tissue valves in younger people wear out quicker because of the more active blood flow in younger people places greater demands on the valve. The risk of death or serious complications from surgical valve replacement is typically from 1% to 5% depending on the health of the patient and the skill of the surgeon. Therefore it is preferred if a valve can be replaced only once. Pediatric valve replacements are difficult because although mechanical valves last better in the younger patient, since the child is still growing, they frequently outgrow their mechanical valve and require replacement of a larger valve with the coordinate required surgical intervention.

Progressive deterioration of tissue valves manifests itself either as obstruction to forward flow through the valve in the open position, i.e. stenosis, or more commonly as tears in the valve leaflets that cause leakage in the closed position, i.e. regurgitation.

U.S. Pat. No. 6,726,715 describes a leaflet for a valve made of fiberous material to form a soft non-mechanical valve. The disadvantage of this valve is that it is synthetic and as such will never assimilate fully into the surrounding tissue. In addition, attachment of this valve is directed using a ring in a planar configuration that risks perivalvular leakage in the same manner as the attachments of mechanical valves.

### Sewing Rings

The valves of the heart separate the heart chambers, and are each mounted in an annulus between them. The annuluses comprise dense fibrous rings attached either directly or indirectly to the atrial and ventricular muscle fibers. In a valve replacement operation, the damaged leaflets are excised and the annulus sculpted to receive a replacement valve. Ideally the annulus presents relatively healthy tissue which can be formed by the surgeon into a uniform ledge projecting into the orifice left by the removed valve. The time and special constraints imposed by surgery, however, often dictate that the shape of the resulting annulus is less than perfect for attachment of a sewing ring. Moreover, the annulus may be calcified as well as the leaflets and complete annular debridement, or removal of the hardened tissue, results in a larger orifice and less declined annulus ledge to which to attach the sewing ring. In short, the contours of the resulting annulus vary widely after the natural valve has been excised.

Conventional placement of the valve is intra-annular, with the valve body deep within the narrowest portion of the annulus to enhance any seal effected by the sewing ring/suture combination and reduce the chance of perivalvular leakage. Surgeons report using at least 30 simple sutures or 20 mattress-type sutures to prevent leakage.

The implantation of a prosthetic heart valve, either a mechanical valve or a bioprosthetic valve (i.e., “tissue” valve), requires a great deal of skill and concentration given the delicate nature of the native heart tissue, the spatial constraints of the surgical field and the criticality of achieving a secure and reliable implantation. It is of equal importance that the valve itself have characteristics that promote a long valve life and that have minimal impact on the physiological makeup of the heart environment.

Given the uneven nature of the annuluses, the design of the sewing ring and the method with which the sewing ring is fixed into place are perhaps the most crucial aspects of prosthetic heart valve implantation.

If the selected size of the ring is slightly too small, the inability of the ring to easily stretch results in undue tension on the tissue and sutures in order to achieve attachment. As a result, a great deal of care and accuracy by the surgeon are needed in the selection of a valve size that precisely matches the valve annulus of the patient. Unfortunately, standard sizing tools are provided in increments based on an overall orifice size, and may not be able to accurately measure a less than optimally formed annulus. The surgeon thus must use informed judgment in selecting an approximate valve size.

U.S. Pat. No. 6,045,576 describes a sewing ring made of silicon rubber with a biocompatible fabric covering at least an outer portion of it. The ring has various configurations including cells, and a specially designed shape for a mitral valve ring. It is described that the sewing ring will be slightly larger than the annulus to provide a close fit within the annulus, stretching the annulus upon placement.

It would be a great boost for valve replacement procedures if a valve could be developed that had the benefits of a tissue valve, and the longevity of a mechanical valve, without the side effects or disadvantages of either. Surgical medicine would also benefit greatly by an improved sewing ring to improve tissue attachment in all valve replacements.

### SUMMARY OF THE INVENTION

The valve developed by the inventor has three main components that account for its success and novelty: the design, the material with which it is made, and the way that it is attached at the site of valve replacement.

The valve is designed to replace a trileaflet valve such as the aortic or pulmonary valves in the human heart. The valve has 3 equally sized leaflets that extend from a valve circumference to a radial center point of the valve, each leaflet contacting or slightly overlapping its two adjacent leaflets. When placed on a flat surface before attaching the valve in the patient, the valve is flat. It can have a sewing ring, which is a firm ring to which the leaflets are attached, and which then attaches approximately at the valvular annulus at the site of valve replacement. The leaflets can be made of a biointegrating material such that over time in the body the leaflets become material similar to or identical to native material found in the body. A preferred such material is exogenous native extracellular matrix material from other mammals.

The circumference of the valve which is defined by the sewing ring is a larger than the circumference of the annulus of the valve lumen where the replacement is to occur. The ratio of the two circumferences (the valve circumference and the annular circumference) is at least greater than 1 and preferably in a range of 1.01 to 1.55. Thus, although the valve is planar outside the body, upon placement at the annulus of the defective valve, it becomes non-planar. Thus, when attached in the annulus, the valve leaflets configure much like a native valve and work to control blood flow like a native valve does.

The advantage of having the sewing ring fit closely to the valvular annulus is that the sewing ring as designed does not have much width and thus does not encroach on the diameter of the valvular annulus. Other valves often have an attachment ring or mechanism that is at least 4 to 5 mm. Where the diameter of the lumen in total is 25 mm, this means that the prosthetic valve encroaches upon the natural annulus and reduces the amount of blood flow allowable once the prosthesis is placed in the annulus. The present invention, however, can have a sewing ring that is a mere 1 mm thick, and thus conserves needed space in the valvular annulus, providing more luminal space for blood flow.

The material used to make the leaflets of the valve is a bio-integrating material, preferably an extracellular matrix material. Although theoretically any extracellular matrix material can be used for this purpose, preferred extracellular matrix materials are exogenous mammalian extracellular matrixes such as those derived from porcine or bovine sources from such tissues as small intestine submucosa (SIS), stomach submucosa (SS), liver basement membrane (LBM), urinary bladder submucosa (UBS), and in general any other extracellular matrix material retrievable from a mammal. See U.S. Pat. No. 5,554,389 (UBS), U.S. Pat. No. 6,099,567 (SS), and U.S. Pat. No. 6,379,710 (LBM), U.S. Pat. No. 4,902,508, U.S. Pat. No. 4,956,178, U.S. Pat. No. 5,275,826, U.S. Pat. No. 5,516,533, U.S. Pat. No. 5,573,784, U.S. Pat. No. 5,711,969, U.S. Pat. No. 5,755,791, U.S. Pat. No. 5,955,110, U.S. Pat. No. 5,968,096, U.S. Pat. No. 5,997,575, and U.S. Pat. No. 6,653,291 (SIS), which are specifically incorporated by reference in their entirety. The advantage of using the extracellular matrix materials from native mammalian sources is that this material is known to regenerate tissue at the site where it is placed in a human or other mammal. Thus, the leaflets become human leaflet tissue after about 3 to 6 months in the human body. The regenerated tissue will be like new tissue with the coordinate lifespan of new tissue, and will not need to be replaced.

In addition, with pediatric patients, the leaflet tissue can grow with the patient and expand as the patient's heart tissue grows to adult proportions, thus eliminating the risk of a second or subsequent surgery to replace the valve.

The third aspect of the valves is the way that the valve is attached in the human. The circumference of the valve is greater than the annulus of the valve being replaced. The valve will have three attached leaflets in a semi-lunar configuration, which configuration is dictated by a circular sewing ring to which the three leaflets are attached, the sewing ring effectively forming the circumference of the valve. The circumference of the valve will be greater than the circumference of the annulus to which it is to be placed. When the valve is placed in the annulus therefore, it is placed in a non-planar configuration so that the circumference fits within the generally annular region. Thus, using either intermittent, or continuous attachment points (such as suture) the valve is attached in a wave-like pattern so that each leaflet has the same high and low attachment points that vary from the plane of the annulus. This attachment means forms leaflets that form a valve in the annulus that will act like a native tissue valve having native tissue leaflets with a rise and fall of leaflet tissue providing for a uni-directional flow of fluid into the heart chamber. This method of attachment also reduces or eliminates the risk of perivalvular leakage because the fit between the valve and the resident annulus is tight and closely conforming. In addition, because the sewing ring of the valve intrudes much less on the lumen of the aorta, after attachment the valve provides the largest possible lumen for blood flow through the region. Preferred attachment is using multiple sutures along the sewing ring, forming attachment of the sewing ring in an up and down configuration along the annular region to make the ring fit generally where the annulus of the defective valve was and to direct three-dimensional structural formation of the leaflets which structure directs them to function as true native leaflets do in healthy native valves.

The sewing ring for use in the valve can be made of extracellular matrix or it can be made of a more conventional material such as metal, nitinol, other shape memory activatable materials, plastic, silicon, rubber or polymers, or these materials wrapped in extracellular matrix. In any case, the leaflets are wrapped around or otherwise attached to the sewing ring. Where the sewing ring is constructed of extracellular matrix, the extracellular matrix can be rolled to form several layers on a tubular configuration forming the ring by attachment of the two ends of the rolled material (see FIG. 4A), or it can be stamped from a laminate sheet, thus having several layers of extracellular matrix laminate to form the ring (see FIG. 4C), or a circular or linear strip of material having a width can be sewn, glued, or otherwise attached to itself forming a tear drop like tube that extends for a length and can be attached at the two ends, or extends for a circular distance in a ring formation (see FIG. 4B).

These and other advantages of the valves are described in greater detail below, and it will be clear from the description the several applications, and variations possible with the valve construction.

### The Sewing Ring

In addition, to a novel valve, the inventor has also developed a sewing ring made of extracellular matrix that can be used either with the leaflets described for the trileaflet valve, or with other valves such as other pulmonary, aortic, bicuspid (or mitral) or tricuspid valves. The sewing ring can be used for mechanical or tissue valves. A preferred material for the valve and the sewing ring is extracellular matrix material, but it can also be formed of a metal or alloy and wrapped with extracellular matrix material or wrapped with leaflets made of extracellular matrix material.

The sewing ring is a circular unit made of multilaminate extracellular matrix sheet, or a tightly configured roll of extracellular matrix forming a solid tube that is formed into a ring and attached to itself by an appropriate attachment means such as suture, or a tear drop shaped tube of a strip of extracellular matrix material folded upon itself. The multilaminate sewing ring can be cut out of a multilaminate sheet in a ring formation of an appropriate width for the valve being replaced.

In case of an aortic or pulmonary valve replacement the ratio of circumference of the sewing ring to annulus will be in a range from 1.01 to 1.55 so that the ring when attached is nonplanar. In the case of a bicuspid (mitral) or tricuspid valve replacement the ratio of the circumference of the sewing ring to annulus will also be in a range from 1.01 to 1.55. Optimally, a distributor will provide a series of different sizes of sewing rings for a surgeon to choose from during surgery after the defective valve is removed and the true dimensions of the remaining annulus have been determined.

The circumference of the annulus where the sewing ring is to be placed can be in a range from about 2 cm to about 15 cm.

Specifically, the invention is a sewing ring for attaching a prosthetic valve for controlling fluid flow in a lumen, the lumen having an annulus, the sewing ring having a circumference a defined distance greater than a circumference of the annulus, so that upon placement of the sewing ring at the annulus, the attached prosthetic valve is not planar within the lumen. The circumference of the annulus is at least 2 cm. The circumference of the annulus can be in a range from about 2 cm to about 15 cm. The ratio of the circumference of the sewing ring and the annulus is at least greater than 1. The attachment to the prosthetic valve can be accomplished at a minimum of three equally spaced points on the sewing ring and at the annulus. The attachment to the prosthetic valve can be accomplished at six or more equally spaced points on the sewing ring and at the annulus. The attachment to the prosthetic valve can be accomplished at twelve or more equally spaced points on the sewing ring and at the annulus.

The attachment to the prosthetic valve can be accomplished with essentially continual equally spaced attachment points on the sewing ring and at the annulus. The sewing ring can comprise a biointegrating material. The biointegrating material can comprise an extracellular matrix material. The sewing ring can comprise a shape memory activated material.

The sewing ring can comprise a synthetic material. The extracellular matrix material can be a multilaminate or a rolled sheet. The sewing ring can be for an aortic valve, pulmonary valve, mitral valve or tricuspid valve replacement. The extracellular matrix can comprise mammalian extracellular matrix. The mammalian extracellular matrix can be porcine. The porcine extracellular matrix can be selected from the group consisting of small intestine submucosa (SIS), stomach submucosa (SS), liver basement membrane (LBM) and urinary bladder submucosa (UBS).

The invention also contemplates a sewing ring for leaflets of a valve for controlling fluid flow in a lumen having an annulus comprising: a ring of reinforced extracellular matrix in a multilaminate or rolled configuration, the ring being larger than a circumference of the annulus, by a ratio of at least greater than 1, the leaflets attachable to the sewing ring prior to placement of the ring at the annulus, and the sewing ring essentially planar prior to placement at the annulus, and no longer planar after placement at the annulus.

The invention also includes a method of replacing a defective valve for controlling fluid flow in a lumen having an annulus, the method comprising: providing a sewing ring having a circumference larger than a circumference of the annulus, attaching a prosthetic valve to the sewing ring, and attaching the sewing ring to the annulus, wherein the valve is planar before attachment at the annulus, and attaching the sewing ring at the annulus at non-planar attachment points on the annulus.

These and other advantages of the sewing rings are described in greater detail below, and it will be clear from the description the several applications, and variations possible with the sewing ring construction.

Various exemplary embodiments of the invention are described below. Reference is made to these examples in a non-limiting sense. They are provided to illustrate more broadly applicable aspects of the present invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. All such modifications are intended to be within the scope of the claims made herein.

### BRIEF DESCRIPTION OF THE DRAWINGS

FIG. 1 depicts a trileaflet valve in a planar configuration.

FIG. 2 depicts the valve if cut along the sewing ring and opened up.

FIG. 3 depicts a trileaflet valve attached to an annular region in a non-planar configuration along its sewing ring.

FIG. 4A depicts a sewing ring formed of a roll of extracellular matrix material with a cross section depicting the roll; FIG. 4B depicts the cross section of a tear drop tube formed ring; FIG. 4C depicts another sewing ring made of multilaminate sheets of extracellular matrix material showing the cross section of the multilaminate sheet type of sewing ring.

### DETAILED DESCRIPTION OF THE INVENTION

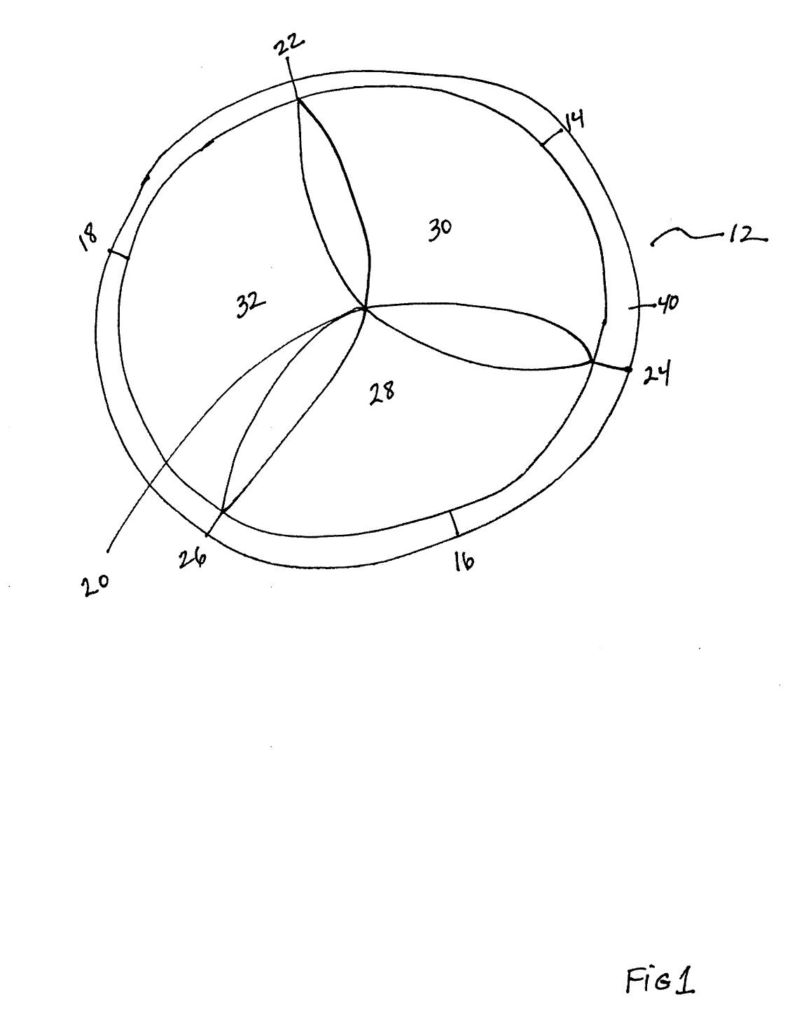

The invention is a valve for controlling fluid flow in a lumen having an annulus. The valve is suitable for replacing an aortic or pulmonary valve. The valve has a sewing ring and three equally sized leaflets. Turning now to the Figures, FIG. 1 depicts the valve as it is before attachment at an annulus. Valve 12 is semi-lunar or essentially circular having an established circumference. Leaflets 28, 30, and 32 overlap adjacent leaflets and extend to the radial center 20 of the valve. Midpoints 14, 16, and 18 of each leaflet define positions where attachment to the annulus is required for securely attaching the valve. Leaflets 28, 30, and 32 are attached to sewing ring 40.

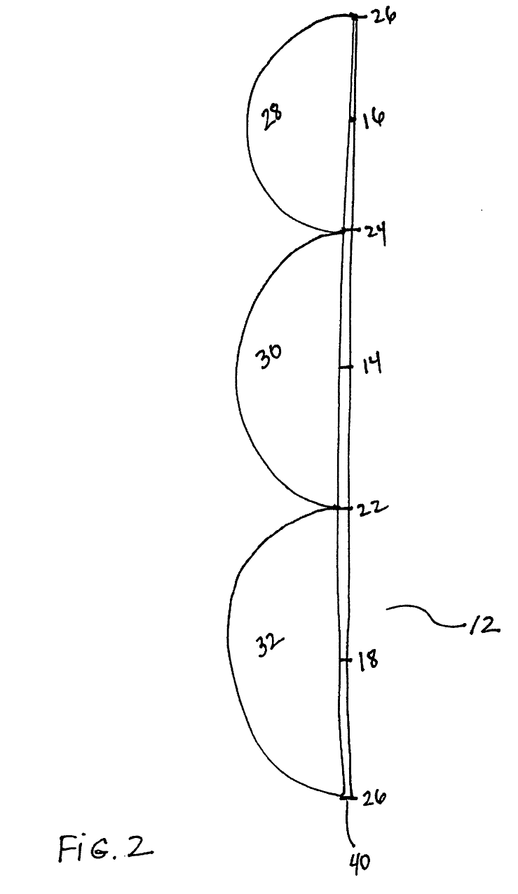

FIG. 2 depicts valve 12 if opened up at a point on the sewing ring 40. Leaflets 28, 30, and 32 are in sequence. Points 26, 22, and 24 mark the points of contact of adjacent leaflets on the sewing ring 40. Midpoints 14, 16, and 18 indicate the middle of each leaflet on the circumferencial ring to which it is attached.

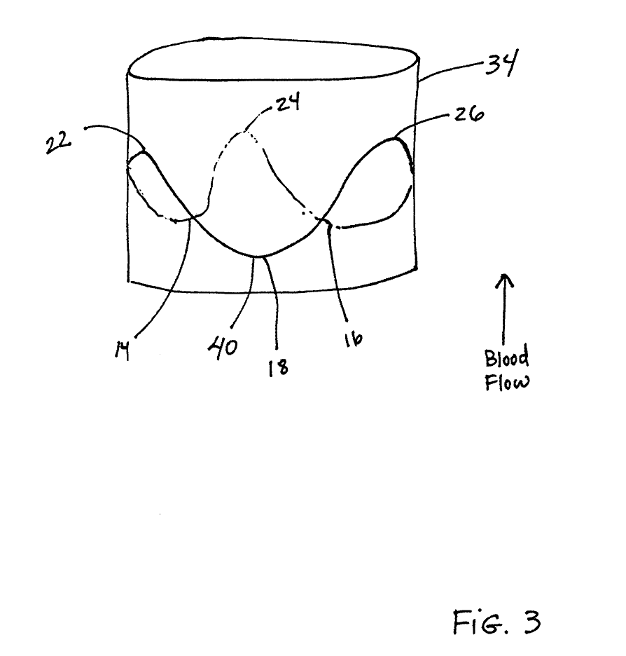

As depicted in FIG. 3, sewing ring 40 is attached to annulus 34, where the midpoints of each leaflet 14, 16, and 18 are indicated, and the points where each leaflet is adjacent to the next leaflet 22, 24, and 26 are indicated. The leaflets themselves are not shown. Blood flow would be in the direction from the bottom of the page to the top of the page through the valve.

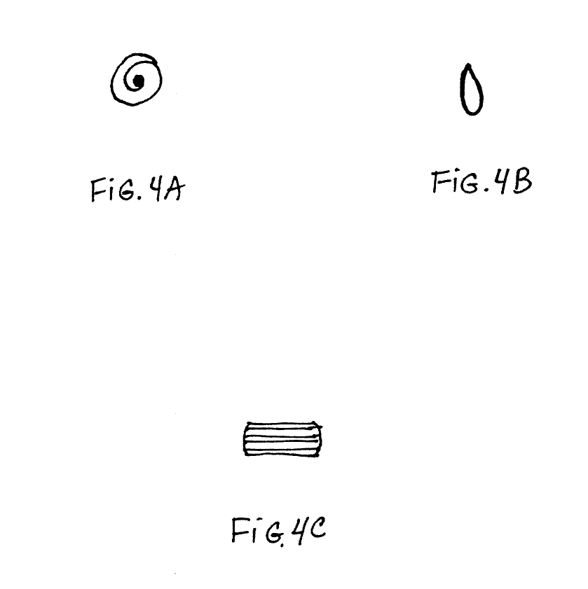

FIG. 4A depicts a sewing ring 40 constructed from a rolled piece of extracellular matrix material. Sewing ring 40 has point of attachment of the two ends of the sewing ring 44. The ring is constructed of a rolled sheet of extracellular matrix having a cross-sectional core 46 as depicted in FIG. 4B. Sewing ring 40 can also be made of a tear drop configuration as depicted in FIG. 4C, where a small strip of extracellular matrix is folded to itself and attached at points 46 with suture or glue or other attachment means. Cross-sectional core 48 depicts the resulting tear-drop configuration of the ring which is attached to itself as in FIG. 4A at point 44, to form ring 40. The sewing ring can also be formed of a laminate sheet stamped out of the laminate sheet. FIG. 4D depicts sewing ring 50 that is stamped out of a larger laminate sheet of for example 7 ply, 8 ply or 10 ply extracellular matrix. Cross-sectional depiction FIG. 4E shows the sheets 52 of the ring 50 in cross section. Ring 50 is continuous because it is stamped out of a plane of laminate sheets of matrix. Each configuration of sewing ring imparts different advantages, and it is contemplated that different valves will be more or less appropriately suited for the two different variations of sewing ring. For example, sewing ring 40 of rolled extracellular matrix has point 44 where the tube is attached to itself. Point 44 would be considered a weak point in the sewing ring, and the ring needs to be attached to itself and the annulus with particular care and reinforcement so that the ring does not yield or break free at point 44. Sewing ring 50 while unitary is non-tubular and attachment of the ring to the annulus will require the attendant care to that aspect of its configuration. It is anticipated that at the very least suture that surrounds both ring 40 and ring 50 will most securely attach the ring to the annulus. Suture through the ring itself may be difficult due to the dense and strong nature of the extracellular matrix material. Suture may be accomplished with simple stitches or mattress stitches depending on the physician's assessment of the situation.

The leaflets can be made of a bio-integrating material such as extracellular matrix material. The extracellular matrix material can be single sheets of extracellular matrix, or multi-laminate sheets, or some other configuration of extracellular matrix that lends itself to the formation of sheet like leaflets. Once in the body the extracellular matrix material soon integrates into the host tissue and the extracellular matrix sheets will become leaflet material. Extracellular matrix material can be harvested and processed as described in U.S. Pat. No. 5,554,389 (UBS), U.S. Pat. No. 6,099,567 (SS), and U.S. Pat. No. 6,379,710 (LBM), U.S. Pat. No. 4,902,508, U.S. Pat. No. 4,956,178, U.S. Pat. No. 5,275,826, U.S. Pat. No. 5,516,533, U.S. Pat. No. 5,573,784, U.S. Pat. No. 5,711,969, U.S. Pat. No. 5,755,791, U.S. Pat. No. 5,955,110, U.S. Pat. No. 5,968,096, U.S. Pat. No. 5,997,575, and U.S. Pat. No. 6,653,291 (SIS), which are specifically incorporated by reference in their entirety.

Attachment of the valve can occur at a minimum of 3 points on the sewing ring, such as points 22, 24, and 26 depicted in FIGS. 1A and 1B. Preferred is at least 6 attachment points corresponding to points 22, 24, 26 and also points 14, 16 and 18. More points in between these equally spaced points can also be used for attachment consistent with the wave-like pattern formed of the valvular circumference (the sewing ring) as it is attached in a regular non-planar configuration at the annular region, and essentially spanning the annulus, although not attaching strickly along what might be called an annular line. Attachment can be by suture using absorbable or permanent sutures. The exact knot tying technique can be selected at the preference of the operating physician.

It is conceivable that attachment of the valve can be accomplished percutaneously without open heart surgery. The valve can be guided to the site of replacement after the defective valve has been removed, and can be systematically stitched or otherwise attached in the annular region along the guidelines of attaching the valve already depicted, using a visualization technique enabling manipulations in the body within the view of a camera that shows the manipulations to the practitioner.

In addition to comprising extracellular matrix, the sewing ring can also comprise metal, or a mixture of metals or alloys such as Nitinol. The sewing ring can also comprise a shape memory activated (SMA) material, also such as Nitinol, or some other SMA. The sewing ring could conceivably be a synthetic or polymeric material, such a silicone, rubber, or plastic. The sewing ring can be constructed like catheter tubing with reinforced plastic having woven support of metal wire embedded within it. In short, the sewing ring can be made of any material suitable for the purpose identified in the definition of a sewing ring. Key functionality of the sewing ring is a flexibility so that the greater circumference of the sewing ring can be placed into the lesser circumference of the annulus in a non-planar attachment configuration successfully.

The invention includes methods that would follow logically from the acts of putting the valves and sewing rings into good use. Thus, the method of replacing a defective valve for controlling fluid flow in a lumen having an annulus comprises providing a valve having three leaflets disposed on a sewing ring having a circumference such that the leaflets overlap or contact any adjacent leaflets and extend radially to a center point in the valve, the leaflets attached to one another on the sewing ring that forms the circumference of the valve, with circumference of the valve larger than the circumference of the annulus, wherein the valve is planar before attachment at the annulus, and attaching the valve at the annulus at non-planar attachment points on the annulus.

A kit can be assembled having a valve. Additionally sewing rings can be provided separately for attaching any number of valves.

The invention includes methods that may be performed using the subject devices or by other means. The methods may all comprise the act of providing a suitable device. Such provision may be performed by the end user. In other words, the “providing” (e.g., a delivery system) merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as is generally known or appreciated by those with skill in the art.

In addition, though the invention has been described in reference to several examples, optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range is encompassed within the invention.

Also, it is contemplated that any optional feature of the inventive variations described may be set forth and claimed independently, or in combination with any one or more of the features described herein. Reference to a singular item, includes the possibility that there are a plurality of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth n the claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of the claim language.

All references cited are incorporated in their entirety. Although the foregoing invention has been described in detail for purposes of clarity of understanding, it will be obvious that certain modifications may be practiced within the scope of the appended claims.

What is claimed is:

1. A sewing ring for attaching a prosthetic valve for controlling fluid flow in a lumen, the lumen having an annulus,
The sewing ring having a circumference a defined distance greater than a circumference of the annulus, so that upon placement of the sewing ring at the annulus, the attached prosthetic valve is not planar within the lumen.

2. The sewing ring of claim 1, wherein a circumference of the annulus is at least 2 cm.

3. The sewing ring of claim 2, wherein a circumference of the annulus is in a range from about 2 cm to about 15 cm.

4. The sewing ring of claim 1, wherein a ratio of the circumference of the sewing ring and the annulus is at least greater than 1.

5. The sewing ring of claim 1, wherein attachment to the prosthetic valve can be accomplished at a minimum of three equally spaced points on the sewing ring and at the annulus.

6. The sewing ring of claim 1, wherein attachment to the prosthetic valve can be accomplished at six or more equally spaced points on the sewing ring and at the annulus.

7. The sewing ring of claim 1, wherein attachment to the prosthetic valve can be accomplished at twelve or more equally spaced points on the sewing ring and at the annulus.

8. The sewing ring of claim 1, wherein attachment to the prosthetic valve can be accomplished with essentially continual equally spaced attachment points on the sewing ring and at the annulus.

9. The sewing ring of claim 1, wherein the sewing ring comprises a biointegrating material.

10. The sewing ring of claim 8, wherein the biointegrating material comprises an extracellular matrix material.

11. The sewing ring of claim 1, comprising a shape memory activated material.

12. The sewing ring of claim 1, comprising a synthetic material.

13. The sewing ring of claim 9, wherein the extracellular matrix material is multilaminate or a rolled sheet.

14. The sewing ring of claim 1, wherein the valve replaces an aortic valve.

15. The sewing ring of claim 1, wherein the valve replaces a valve selected from the group consisting of a pulmonary valve, a mitral valve, and a tricuspid valve.

16. The sewing ring of claim 10, wherein the extracellular matrix material comprises mammalian extracellular matrix.

17. The sewing ring of claim 16, wherein the mammalian extracellular matrix material is porcine.

18. The sewing ring of claim 16, wherein the porcine extracellular matrix material is selected from the group consisting of small intestine submucosa (SIS), stomach submucosa (SS), liver basement membrane (LBM) and urinary bladder submucosa (UBS).

19. A sewing ring for leaflets of a valve for controlling fluid flow in a lumen having an annulus comprising:
a ring of reinforced extracellular matrix in a multilaminate or rolled configuration, the ring being larger than a circumference of the annulus, by a ratio of at least greater than 1,
the leaflets attachable to the sewing ring prior to placement of the ring at the annulus, and
the sewing ring essentially planar prior to placement at the annulus,
and no longer planar after placement at the annulus.

20. A method of replacing a defective valve for controlling fluid flow in a lumen having an annulus, the method comprising:
providing a sewing ring having a circumference larger than a circumference of the annulus, attaching a prosthetic valve to the sewing ring, and attaching the sewing ring to the annulus, wherein the valve is planar before attachment at the annulus, and attaching the sewing ring at the annulus at non-planar attachment points on the annulus.
